(19) United States
(12) Patent Application Publication
(10) Pub. No.: US 2009/0144903 A1
(43) Pub. Date: Jun. 11, 2009

(54) CPR FACILITATING MATTRESS

(76) Inventors: Andrew B. Delvaux, Green Bay, WI (US); Joshua J. Dykla, Livonia, MI (US); Christopher J. Rivet, Grand Blanc, MI (US); Matthew T. Trombley, Concord, MI (US); Ryan J. Gilbert, Houghton, MI (US); Kristin Cauley, Oshkosh, WI (US); Kyle J. Marsh, Houghton, MI (US); Dennis Jensen, Olympia, WA (US)

Correspondence Address: MICHAEL BEST & FRIEDRICH LLP, 100 E WISCONSIN AVENUE, Suite 3300, MILWAUKEE, WI, 53202, US

(21) Appl. No.: 11/951,501

(22) Filed: Dec. 6, 2007

(51) Int. Cl. A61G 7/00; A61B 5/02

(52) U.S. Cl. 5/617; 600/481

(57) ABSTRACT

A mattress assembly that includes a lower portion for support of a patient's legs, an upper portion for the support of the patient's torso, and an evacuation assembly. The upper portion including an enclosure defining an interior space and a compressible material within the interior space. The evacuation assembly including a vacuum pump communicating with the interior space and operable to evacuate the interior space and compress the compressible material such that the upper portion of the mattress supporting the patient's torso is lowered with respect to the lower portion of the mattress supporting the patient's legs, and such that the upper portion of the mattress becomes stiffer to facilitate CPR on the patient.

### BACKGROUND

The present invention relates to a mattress that maintains the comfort of a patient, but is also quickly adaptable to facilitate cardiopulmonary resuscitation in the event the patient goes into cardiac arrest.

### SUMMARY

In one embodiment, the invention provides a mattress assembly comprising a lower portion for the support of a patient's legs, an upper portion for the support of the patient's torso, the upper portion including an enclosure defining an interior space and a compressible material within the interior space and an evacuation assembly including a vacuum pump communicating with the interior space and operable to evacuate the interior space and compress the compressible material such that the upper portion of the mattress supporting the patient's torso is lowered with respect to the lower portion of the mattress supporting the patient's legs, and such that the upper portion of the mattress becomes stiffer to facilitate CPR on the patient.

In another embodiment the invention provides a method for operating a control system for a mattress assembly having a lower portion adapted to support a patient's legs and an upper portion adapted to support the patient's torso, the method comprising providing a compressible material within the upper portion, enclosing the compressible material within an interior space of an enclosure, placing a vacuum pump in communication with the interior space, monitoring the cardiac condition of a patient supported by the mattress, generating a signal in response to detecting conditions consistent with cardiac arrest in the patient, and initiating an alarm and operating the vacuum pump in response to the signal, the vacuum pump evacuating the interior space of the enclosure and compressing the compressible material to stiffen the upper portion of the mattress assembly and facilitate CPR on the patient.

In another embodiment the invention provides a method of retro-fitting an evacuation assembly to a known mattress, the method comprising providing a mattress for supporting a patient, creating a cavity in the mattress, providing a compressible material, containing the compressible material in an enclosure, installing the compressible material and enclosure in the cavity, and communicating a vacuum pump with the enclosure, wherein the vacuum pump may be actuated to evacuate the enclosure and compress the compressible material to stiffen the mattress and facilitate CPR on the patient.

Other aspects of the invention will become apparent by consideration of the detailed description and accompanying drawings.

### BRIEF DESCRIPTION OF THE DRAWINGS

FIG. 1 illustrates an exemplary bed including a mattress according to the present invention.

FIG. 2 is an exploded view of a portion of the mattress.

FIG. 3 is an exploded view of a portion of an alternative mattress construction.

FIG. 4 is a cross sectional view of the mattress in an at-rest condition.

FIG. 5 is a cross sectional view of the mattress in a CPR-ready condition.

FIG. 6 is an exploded view of another alternative mattress construction.

FIG. 7 is a perspective view of a portion of the mattress illustrated in FIG. 6.

FIG. 8 is a schematic of a control system for the mattress constructions.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

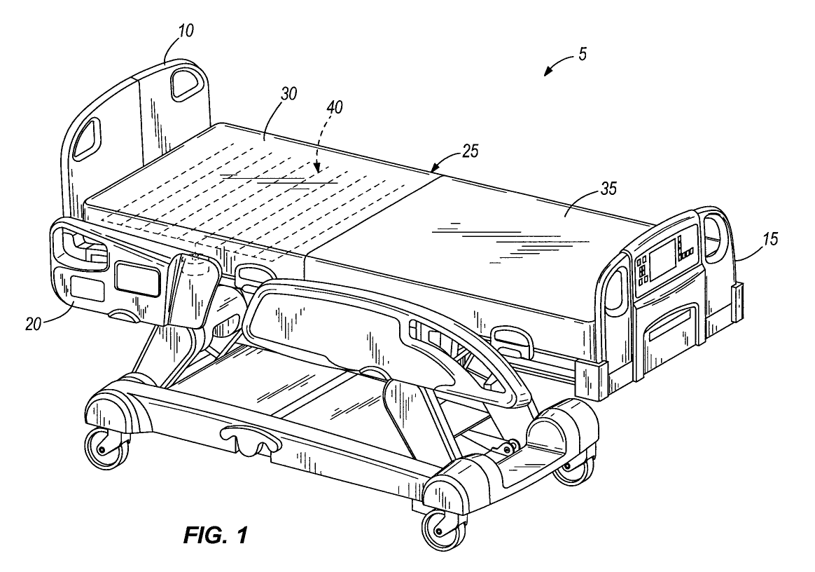

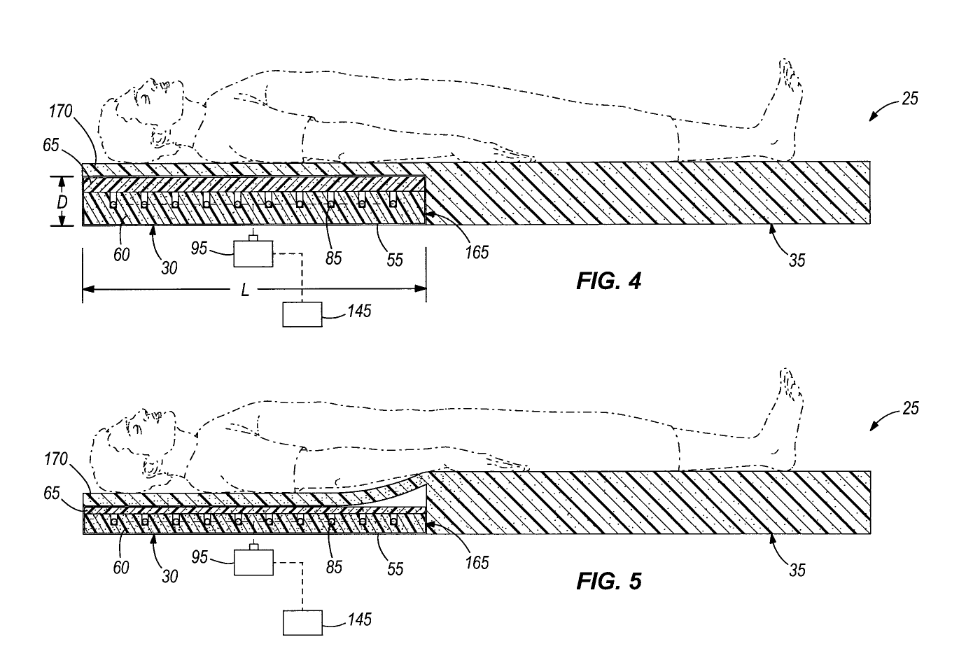

FIGS. 1-5 illustrate a first embodiment of a bed 5 as may be used in a hospital, clinic, urgent care facility, rehabilitation facility, hospice, home, or other facility or location on which a patient (shown in FIGS. 4 and 5) may be situated. With reference to FIG. 1, the bed 5 includes a headboard 10, footboard 15, and support structure or frame 20 extending between the headboard 10 and footboard 15. A mattress 25 is supported by the support structure 20, and includes an upper portion 30, a lower portion 35, and an evacuation assembly 40. The upper portion 30 of the mattress 25 supports the patient's head and torso, and the lower portion 35 supports the patient's legs. The upper and lower portions 30, 35 of the mattress 25 are pivotable with respect to each other, and the bed support structure 20 may be actuable to adjust the upper and lower portions 30, 35 of the mattress 25 at relative angles to maximize the comfort of the patient. Generally, bed controls are used to adjust the angles of the upper and lower portions 30, 35 of the mattress 25, and such bed controls can be of a power-assisted or manual variety.

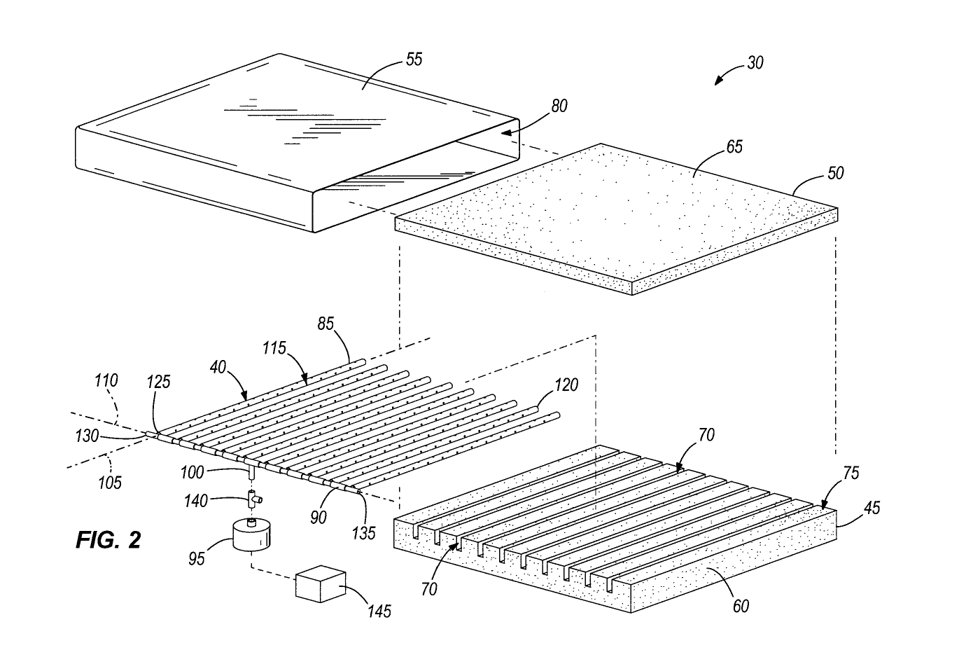

With reference to FIG. 2, the upper portion 30 of the mattress 25 includes a first compressible material 45, a second compressible material 50, and an enclosure 55. The term “compressible material” as used herein includes materials that contain air, such that the volume of the material can be reduced by evacuating the air. One example of a compressible material is foam, and the first and second compressible materials 45, 50 illustrated in FIG. 2 take the form of first and second foam structures 60, 65.

The first foam structure 60 defines a plurality of generally parallel open channels 70. As used herein, the term “open channel” refers to channels 70 having three defined sides and a fourth side open, or channels 70 having at least a portion of one side open. In other embodiments, the open channels 70 may be a variety of recessed contours, such as an arc or v-shape. In this embodiment, the open channels 70 open upwardly and intersect with an upper planar surface 75 of the first foam structure 60, but in other embodiments (e.g., the second embodiment disclosed below) the channels may open in a different direction, such as downwardly. In other embodiments, the channels may be closed, in which case the channels 70 would take the form of tunnels or bores through the first foam structure 60.

The second foam structure 65 is in the form of a generally flat mat extending over the upper planar surface 75 of the first foam structure 60 to cover and close the open channels 70. The enclosure 55 is constructed of a flexible, durable material, and defines an interior space 80. The first and second foam structures 60, 65 are contained within the interior space 80, and the enclosure 55 is air-tightly sealed around the first and second foam structures 60, 65.

The evacuation assembly 40 includes a plurality of tubes 85, a manifold 90, a vacuum pump 95, and a connecting conduit 100. Each of the tubes 85 has a tube longitudinal axis 105 and the manifold 90 has a manifold longitudinal axis 110. The longitudinal axes 105, 110 of the tubes 85 and manifold 90 define the lengthwise direction of the respective tubes 85 and manifold 90. The longitudinal axes 105 of the tubes 85 are generally parallel to each other and generally perpendicular to the longitudinal axis 110 of the manifold 90 in the illustrated embodiment. Each tube 85 includes a plurality of apertures or holes 115 spaced along its length. One end 120 of each tube 85 is closed, and the opposite end 125 fluidly communicates with the manifold 90. Both ends 130, 135 of the manifold 90 are closed. The each tube 85 is received within one of the open channels 70 and is recessed into the first foam structure 60 below the upper planar surface 75. The plurality of tubes 85 and the manifold 90 are contained within the interior space 80 of the enclosure 55, with the connecting conduit 100 extending through the enclosure 55.

The connecting conduit 100 communicates between the vacuum pump 95 (outside the interior space 80 of the enclosure 55) and the manifold 90 (inside the interior space 80 of the enclosure 55) to facilitate the evacuation of air from the interior space, thereby compressing and stiffening the upper portion 30. The enclosure 55 is air-tightly sealed around the connecting conduit 100. The vacuum pump 95 evacuates air from the interior space via the apertures 115 of the tubes 85 fluidly coupled to the manifold 90. As used herein, the term “evacuate” means to draw air from, and does not necessarily require the complete evacuation of (i.e., the formation of a perfect vacuum within) the interior space. In the illustrated embodiment, the connecting conduit 100 intersects the manifold 90 near the center of the manifold's longitudinal extent. In other embodiments, the connecting conduit 100 may connect closer to or at one of the ends 130, 135 of the manifold 90. The connecting conduit 100 may include means for selectively cutting off or resisting air flow into or out of the interior space 80 of the enclosure 55 to selectively maintain the upper portion 30 of the mattress 25 in an at-rest inflated condition or an evacuated condition (such conditions being described in more detail below). The means for resisting air flow may include in some embodiments a quick-disconnect coupling, a regulator, a check valve, or the like.

The connecting conduit 100 may also include a T-shaped joint 140 that facilitates the connecting of an alternate vacuum source (such as a central vacuum source in a hospital or an additional vacuum pump) or a positive airflow source (such as a blower or positive pressure pump) to assist in evacuating and inflating the interior space 80. In other embodiments, the T-shaped joint 140 may simply provide selective communication between the interior space 80 and the atmosphere during re-inflation of the interior space 80 following evacuation so that air flows directly into the interior space 80 in addition to or instead of only through the vacuum pump 95. When the vacuum pump 95 is turned off or disengaged, the foam returns to its original shape and draws air into the enclosure 55 or a positive pressure pump may be used to assist the return of the foam to its original at-rest shape and dimensions.

A power source 145 for the vacuum pump 95 may include a portable power source that is part of the evacuation assembly. Such portable power source may be, for example, a battery that is coupled to the support structure 20 of the bed 5. The portable power source may be carried on the frame 20 of the bed 5 or within the mattress 25 itself to provide a modular bed or mattress that can be moved around within a facility without regard to the location of external, fixed-location or portable power hookups. In other embodiments, the power source 145 may include an electrical cord coupled to an electrical outlet, in which case the power source 145 would be provided separately from the evacuation assembly 40.

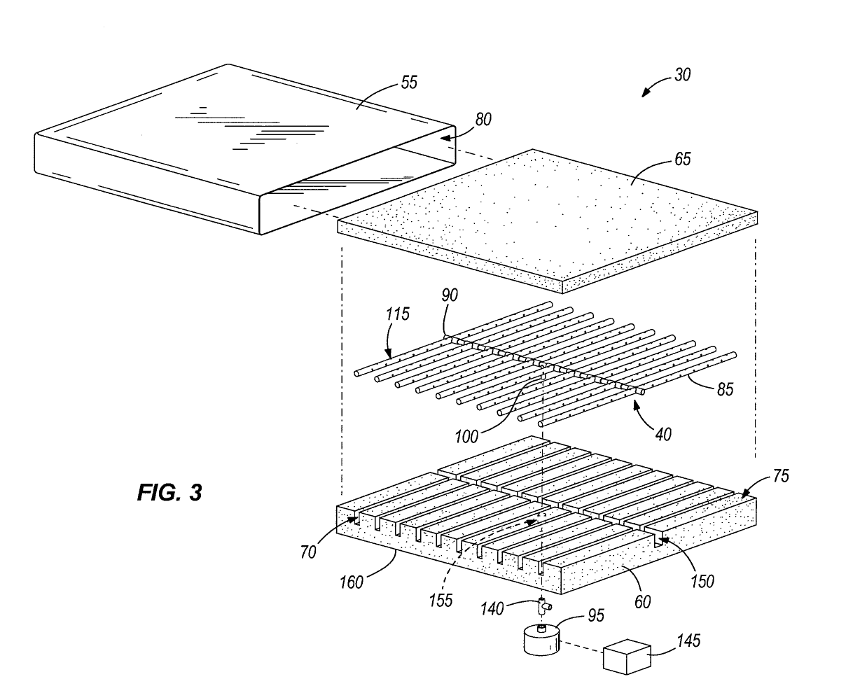

FIG. 3 illustrates an alternative construction in which the tubes 85 are shorter and extend in opposite directions from the manifold 90, such that the manifold 90 extends down the middle of the parallel array of tubes. An additional, perpendicular open channel 150 is provided in the first foam structure 60 to accommodate the manifold 90 in this embodiment. The connecting conduit 100 extends through a hole 155 in a bottom 160 of the first foam structure 60 to communicate with the vacuum pump 95 (either directly or through a joint 140 as discussed above). In all embodiments, the manifold 90 may comprise a relatively stiff tube or a more flexible tube or hose if it is desired to provide an upper mattress portion that can be manipulated into non-planar shapes.

In some embodiments, the first foam structure 60 may be constructed of known hospital bed foam, and the second foam structure 65 may be constructed of memory foam. The second foam structure 65 may have a compressibility greater than the first foam structure 60. In some embodiments, the enclosure 55 may be constructed of 3.5 mil plastic sheeting. In some embodiments, the tubes 85 may be constructed of plastic tubing material, and the holes 115 may be drilled or otherwise formed at 1.5 inch intervals along the tubes' lengths to provide even and quick evacuation of the interior space 80. In some embodiments, the vacuum pump 95 capacity and foam types may be selected to achieve deflation of the interior space 80 in less than 22 seconds, with a compression efficiency greater than 88%. Most compression in the evacuation assembly 40 will take place within the memory foam, which may have a compression efficiency of about 92.6%.

In some embodiments, the first and second foam structures 60, 65 may be dimensioned to provide the entire upper portion 30 of the mattress 25. In other embodiments (as illustrated in FIGS. 4 and 5), the first and second foam structures 60, 65 may be dimensioned to fit within a cavity 165 in the back of the upper portion 30 of the mattress 25. More specifically, with reference to FIG. 4, the cavity 165 may be about 32 inches in length L, 35 inches wide (i.e., into the page), and 5 inches in depth D. A relatively thin front portion 170 of the mattress 25 remains over the cavity 165, between the foam structures 60, 65 and the patient. In such example, the first foam structure 60 may be constructed of hospital bed foam and be about 3.5 inches thick, and the second foam structure 65 may be constructed of shape memory foam and be about 1.5 inches thick (such that the combined thickness of the first and second foam structures 60, 65 fills the 5 inch depth of the cavity 165). The evacuation assembly 40 (including in some cases the vacuum pump 95 and power source 145) may be contained within the upper portion 30 of the mattress 25.

With reference to FIG. 4, during normal, at-rest operation, the upper and lower portions 30, 35 of the mattress 25 provide a substantially planar surface upon which a patient rests. Depending on the functionality of the support structure 20, the upper and lower portions 30, 35 of the mattress 25 can be manipulated to provide a non-planar support surface that is comfortable for the patient. With reference to FIG. 5, upon cardiac arrest or when appropriate during respiratory arrest, medical personnel may flatten the bed support structure 20, and evacuate the interior space 80 of the enclosure 55 to compress the first and second foam structures 60, 65 and increase the hardness of the mattress 25 under the patient's torso to facilitate cardiopulmonary resuscitation (CPR). The lower portion 35 of the mattress 25 remains at the ordinary at-rest thickness such that the patient's legs are effectively raised with respect to the patient's heart to further support the CPR effort.

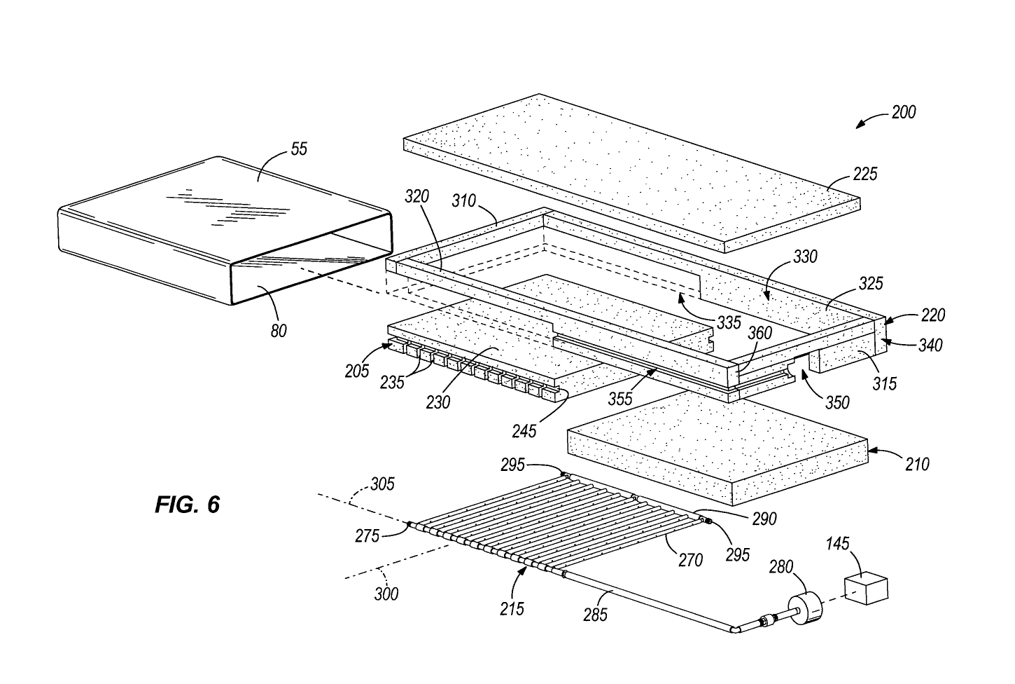

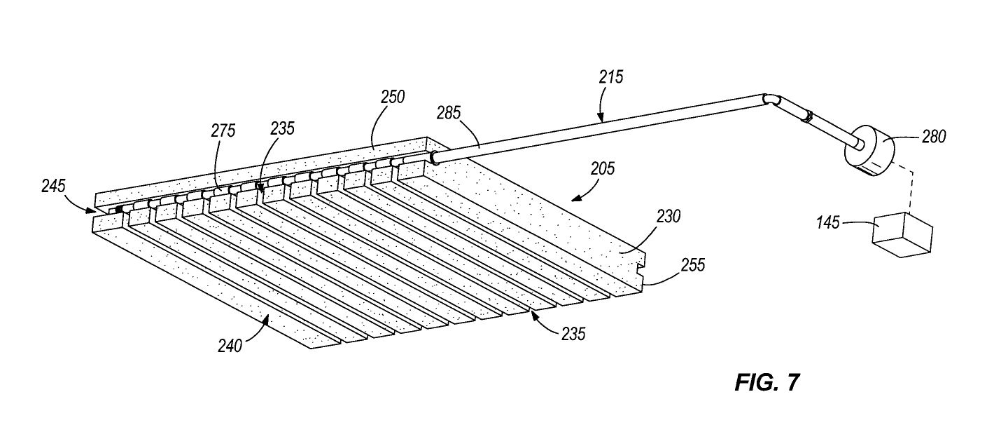

FIGS. 6 and 7 illustrate a second embodiment of the invention. Parts that are common or substantially similar with those in the first embodiment are identified with the same reference numerals as used with reference to the first embodiment. Also, it is possible that various aspects of the first and second embodiments may be included in the other embodiment, so aspects of the embodiments should not be read as limited to the embodiment simply because they are explained with respect to the embodiment. As with the first embodiment, this second embodiment includes a mattress assembly 200 (for use in a bed such as illustrated in FIG. 1) having upper and lower portions 205, 210, and an evacuation assembly 215. The mattress assembly 200 in this embodiment also includes a mattress frame or perimeter 220 surrounding the upper and lower mattress portions 205, 210, and a full length thin foam structure 225 overlaying the upper and lower portions 205, 210 of the mattress 200.

The lower portion 210 of the mattress 200 is substantially the same as that of the first embodiment. The upper portion 205 of the mattress 200 in this embodiment includes a single compressible material structure 230 which in the illustrated embodiment is a foam structure constructed of hospital mattress foam. The foam structure 230, as best shown in FIG. 7, includes a plurality of generally parallel downwardly-opening channels 235 similar to the upwardly-opening channels 70 in the first embodiment except that the downwardly-opening channels 235 intersect a planar surface 240 defined by the bottom of the foam structure 230. In other constructions, the downwardly-opening channels 235 may open in a different direction or may take the form of closed channels or tunnels in the single foam structure 230. The foam structure 230 also includes open side channels 245 along opposite sides 250, 255, generally perpendicular to and intersecting the downwardly-opening channels 235 and generally vertical planes defined by the first and second sides 250, 255 of the foam structure 230.

As with the evacuation of the first embodiment, the evacuation assembly 215 in this embodiment includes a plurality of tubes 270, a first manifold 275, a vacuum pump 280, and a connecting conduit 285. The evacuation assembly 215 of this embodiment further includes a second manifold 290 that has closed ends 295. As with the first embodiment, the tubes 270 and manifolds 275, 290 (FIG. 6) have longitudinal axes 300, 305, and the longitudinal axis 300 of each tube 270 is generally perpendicular to the longitudinal axis 305 of each manifold 275, 290. Each of the tubes 270 fluidly communicates between the first and second manifolds 275, 290. The tubes 270 are received within the downwardly-opening channels 235 and the first and second manifolds 275, 290 are received within the open side channels 245. The foam structure 230, tubes 270, and manifolds 275, 290 are all contained within the interior space 80 of the enclosure 55. Having manifold tubes 275, 290 at each end of the tubes 270 may create a more stable structure for the upper portion 205 of the mattress 200, and may also better resist lateral (i.e. along the longitudinal axes 300 of the tubes 270) shrinking of the upper portion foam structure 205 than the evacuation assembly 40 of the first embodiment.

With respect to FIG. 6, the mattress frame 220 includes upper end 310, lower end 315, and side portions 320, 325, an inwardly-facing surface 330 of the mattress frame 220 defines a rectangular space 335, and an outwardly-facing surface defines an exterior surface 340 of the overall mattress 200. As used herein, the term “mattress envelope” means within a space enclosed by the outer surface 340 of the overall mattress 200 (i.e., the outer surface 340 of the mattress frame 220 in this embodiment).

The mattress frame 220 may in some constructions be made of hospital bed foam or a slightly stiffer foam or other resilient material to provide stiffer support at the edges of the mattress 200 for patients seated on the side portions, for example. Also, the frame 220 resists deflection during evacuation of the interior space 80, which helps contain the patient on the mattress 200 as the upper portion 205 is evacuated and shrunk. This reduces the likelihood of a patient rolling off the mattress 200 and bed 5.

The lower end portion 315 of the mattress frame 220 includes a cut out 350 sized and shaped to receive the vacuum pump 280, although in other embodiments the cut out 350 for the vacuum pump 280 may be provided in any other portion of the mattress frame 220. The lower end portion 315 and one of the side portions 320 of the mattress frame 220 include a perimeter channel 355 (which in other embodiments may be a closed channel or tunnel) that is sized and shaped to receive the connecting conduit 285. The perimeter channel 355 extends along the end portion 315 of the mattress frame 220, around a corner 360 of the mattress frame 220, and along the side portion 320 of the mattress frame 220. The connecting conduit 285 is substantially L-shaped to follow the perimeter channel 355 around the corner 360 of the mattress frame 220. A t-shaped joint similar to joint 140 described above may also be employed in this embodiment.

The vacuum pump 280 and connecting conduit 285 may be described as being positioned within the mattress envelope in this embodiment. Positioning the vacuum pump 280 and connecting conduit 285 within the mattress envelope provides a compact, modular mattress design that may enable the entire mattress and evacuation assembly 200 to be installed in place of an existing, conventional mattress on an existing bed with little or no modifications to the bed.

The second embodiment functions substantially the same way as the first embodiment. Namely, when at rest, the upper and lower mattress portions 205, 210 define a generally planar surface to support a patient. When the vacuum pump 280 is engaged, it evacuates the interior space 80, and the single foam structure 230 is compressed to increase the hardness of the mattress 200. When the vacuum pump 280 is turned off or disengaged, the foam returns to its original shape and draws air into the enclosure 55 or a positive pressure pump may be used to assist the return of the foam to its original at-rest shape and dimensions.

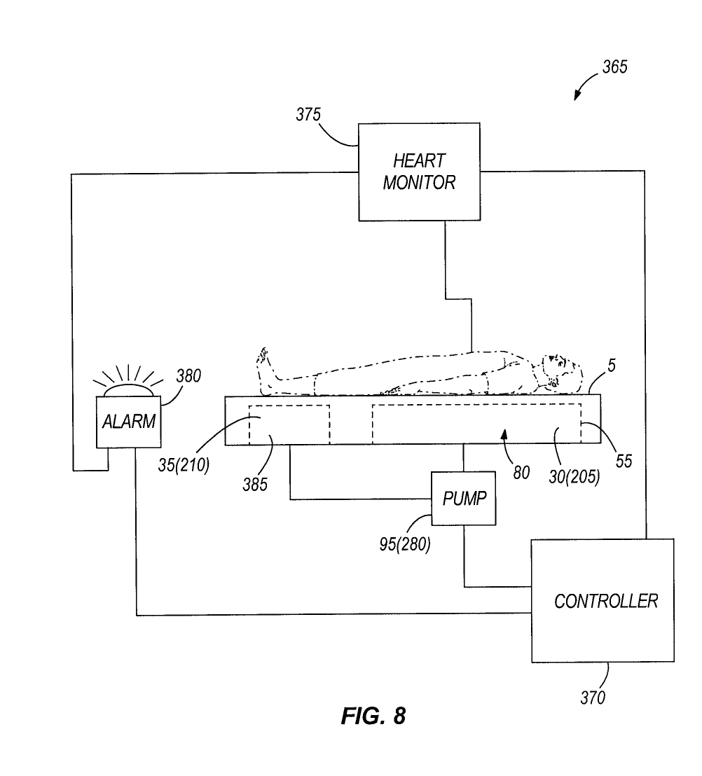

Turning now to FIG. 8, one potential control system 365 for use with either embodiment may be employed to automatically engage the vacuum pump in the event the patient experiences cardiac arrest. The control system 365 includes a processor or controller 370 electronically (wired or wirelessly) communicating with a heart monitor 375, the vacuum pump, and an alarm 380. The heart monitor 375 is attached to the patient and generates a signal in response to conditions consistent with the patient experiencing cardiac arrest. The controller 370 automatically initiates operation of the vacuum pump in response to receiving the signal from the heart monitor 375. The controller 370 may also actuate the bed controls to flatten the mattress if the bed 5 is in a non-flat condition when the heart monitor 375 generates the signal.

Also upon sensing conditions consistent with cardiac arrest, the controller 370 sets off the alarm 380 (which may be visual, audible, or a combination of the two) to alert medical personnel or other attendants of the situation. In an alternative embodiment, the alarm 380 may be set off by a controller associated with the heart monitor 375 rather than by the controller 370. Automatically flattening the bed 5 and initiating operation of the vacuum pump may improve the effectiveness of CPR, because conditions such as flattening the mattress and engaging the vacuum pump to stiffen the upper portion of the mattress will be initiated during the medical personnel response time, rather than after the medical personnel arrive.

In another construction (and as illustrated in FIG. 8), the lower portion of the mattress may be modified to include a fluid bladder or pillow 385. As the vacuum pump evacuates the interior space 80 of the enclosure 55 under the patient's torso, the evacuated fluid is diverted to the fluid bladder 385 under the patient's legs. As the fluid bladder 385 inflates, it raises the patient's legs to allow better blood flow back to the heart to help improve the quality of chest compressions.

In other embodiments, the vacuum pump may be within the interior space 80, provided that it communicates with the atmosphere outside of the interior space 80. The enclosure 55 is air tightly sealed around the components that are within the interior space 80, such that the interior space 80 communicates with the atmosphere only through the vacuum pump.

In other constructions, the evacuation assembly may be oriented 90° clockwise or counter-clockwise within a plane defined by the longitudinal axes of the tubes.

Various features and advantages of the invention are set forth in the following claims.

What is claimed is:

1. A mattress assembly comprising:
a lower portion for the support of a patient's legs;
an upper portion for the support of the patient's torso, the upper portion including an enclosure defining an interior space and a compressible material within the interior space; and
an evacuation assembly including a vacuum pump communicating with the interior space and operable to evacuate the interior space and compress the compressible material such that the upper portion of the mattress supporting the patient's torso is lowered with respect to the lower portion of the mattress supporting the patient's legs, and such that the upper portion of the mattress becomes stiffer to facilitate CPR on the patient.

2. The mattress assembly of claim 1, wherein the compressible material includes at least one foam structure.

3. The mattress assembly of claim 2, wherein the evacuation assembly includes a plurality of tubes within the at least one foam structure, the plurality of tubes communicating with the vacuum pump such that air is evacuated from the interior space through the plurality of tubes under the influence of the vacuum pump.

4. The mattress assembly of claim 3, wherein the evacuation assembly further includes a manifold communicating between the vacuum pump and the plurality of tubes to distribute suction from the vacuum pump substantially evenly to the plurality of tubes.

5. The mattress assembly of claim 3, wherein the at least one foam structure includes open channels in which the plurality of tubes are received.

6. The mattress assembly of claim 5, wherein the open channels open downwardly.

7. The mattress assembly of claim 5, wherein the open channels open upwardly.

8. The mattress assembly of claim 7, wherein the at least one foam structure includes a first foam structure defining the upwardly-opening channels and a second foam structure extending across the open channels.

9. The mattress assembly of claim 8, wherein the second foam structure has greater compressibility than the first foam structure.

10. The mattress assembly of claim 8, wherein the second foam structure includes a memory foam.

11. The mattress assembly of claim 1, wherein the mattress includes outer surfaces defining a mattress envelope; and wherein substantially the entire evacuation assembly is contained within the mattress envelope.

12. The mattress assembly of claim 1, further comprising: a mattress frame extending around the upper and lower portions; wherein an outer surface of the mattress frame defines a mattress envelope; and wherein the mattress frame includes a cut out in which the vacuum pump is received.

13. The mattress of claim 12, wherein the mattress frame resists deflection during evacuation of the interior space to resist a patient rolling off the mattress.

14. The mattress assembly of claim 12, wherein the evacuation assembly includes a connecting conduit communicating between the vacuum pump and the interior space; wherein the mattress frame includes a perimeter channel; and wherein the connecting conduit is received in the perimeter channel.

15. The mattress assembly of claim 14, wherein the cut out is in an end portion of the mattress frame; wherein the perimeter channel extends along the end portion of the mattress frame, around a corner of the mattress frame, and along a side portion of the mattress frame; and wherein the connecting conduit is substantially L-shaped to follow the perimeter channel around the corner of the mattress frame.

16. The mattress assembly of claim 12, wherein the evacuation assembly includes a transportable power source within the mattress frame and within the mattress envelop, the transportable power source being movable with the mattress assembly and providing power to the vacuum pump.

17. The mattress assembly of claim 1, further comprising: a control system including a monitor to generate a signal in response to detecting conditions consistent with cardiac arrest in the patient; and a controller initiating operation of the vacuum pump in response to receiving the signal from the monitor.

18. The mattress assembly of claim 1, wherein the evacuation assembly includes a T-shaped joint communicating between the vacuum pump and the interior space; wherein the T-shaped joint facilitates communicating an airflow source in addition to the vacuum pump with the interior space; and wherein the airflow source provide at least one of atmospheric air and forced air to the interior space to assist at least one of evacuation and inflation of the compressible material.

19. The mattress assembly of claim 1, wherein the lower portion includes a fluid bladder fluidly connected to the enclosure; and wherein evacuated fluid from the enclosure is used to inflate the fluid bladder to raise the patient's legs.

20. A method for operating a control system for a mattress assembly having a lower portion adapted to support a patient's legs and an upper portion adapted to support the patient's torso, the method comprising:
providing a compressible material within the upper portion;
enclosing the compressible material within an interior space of an enclosure;
placing a vacuum pump in communication with the interior space;
monitoring the cardiac condition of the patient supported by the mattress;
generating a signal in response to detecting conditions consistent with cardiac arrest in the patient; and
initiating an alarm and operating the vacuum pump in response to the signal, the vacuum pump evacuating the interior space of the enclosure and compressing the compressible material to stiffen the upper portion of the mattress assembly and facilitate CPR on the patient.

21. The method of claim 20, further comprising: supporting the mattress assembly with a bed; and flattening the bed in response to the signal.

22. The method of claim 20, further comprising: providing a controller; and receiving the signal with the controller; wherein initiating an alarm and operating the vacuum pump are performed by the controller in response to receiving the signal.

23. A method of retrofitting an evacuation assembly to a known mattress, the method comprising:
providing a mattress for supporting a patient;
creating a cavity in the mattress;
providing a compressible material;
containing the compressible material in an enclosure;
installing the compressible material and enclosure in the cavity; and
communicating a vacuum pump with the enclosure;
wherein the vacuum pump may be actuated to evacuate the enclosure and compress the compressible material to stiffen the mattress and facilitate CPR on the patient.

24. The method of claim 23, wherein the mattress includes a thin portion that remains over the cavity between the enclosure and the patient.

25. The method of claim 23, further comprising: supporting the mattress assembly with a bed; and providing a power source transportable with the bed, wherein the power source supplies power to the vacuum pump.

26. The method of claim 23, further comprising: providing a plurality of tubes within the enclosure; and fluidly coupling the tubes to the vacuum pump.

27. The method of claim 26, further comprising: providing a manifold in fluid communication between the vacuum pump and the plurality of tubes; providing a T-shaped joint in fluid communication between the vacuum pump and the manifold; connecting an alternative airflow source to the T-shaped joint; and using the alternative airflow source to facilitate inflating the enclosure through the T-shaped joint with one of atmospheric air and forced air.

28. The method of claim 26, further comprising: providing channels in the compressible material and installing the plurality of tubes recessed within respective channels of the compressible material.

29. The method of claim 26, further comprising: containing the evacuation assembly and vacuum pump within an envelope of the mattress.
